(19) United States
(12) Patent Application Publication
(10) Pub. No.: US 2026/0248620 A1
(43) Pub. Date: Aug. 27, 2026

(54) METHOD AND APPARATUS FOR REDUCING HUMAN VERTEBRAL BODY SUBSIDENCE USING VARIABLE SURFACE AREA INTERBODY CAGES CORRELATED TO LOCALIZED BONE DENSITY MEASUREMENTS

(71) Applicant: Acuity Surgical Devices, LLC, Irving, TX (US)

(72) Inventors: John R. Davidson, Westlake, TX (US); Bryan M. Cowan, Dallas, TX (US); Charles R. Forton, Celina, TX (US); Antonio D. Valdevit, Effort, PA (US)

(21) Appl. No.: 19/648,899

(22) Filed: Apr. 15, 2026

Related U.S. Application Data
(62) Division of application No. 18/031,307, filed on Nov. 10, 2022.
(60) Provisional application No. 63/280,246, filed on Nov. 17, 2021.

(51) Int. Cl. A61F 2/44; A61F 2/30

(52) U.S. Cl. CPC A61F 2/447; A61F 2002/30056; A61F 2002/30593; A61F 2002/3092

(57) ABSTRACT

An improved interbody spinal implant which takes into account specific patient variables. During the preoperative phase an interactive CT radiodensity scan of localized portions of the vertebrae is used to determine whether the template for a preselected implant will minimize subsidence based on the Hounsfield Unit score and a corresponding calculated Hounsfield Parameter value generated by the scan for the contact surface area of the selected implant. If not, a template for another selected implant having different medial and lateral dimensions is selected and placed over the interactive CT scan. If acceptable Hounsfield Parameters are generated this means acceptable minimal subsidence will occur. The surgeon may then evaluate the size of the bone graft window or the implant opening permitting the surface contact area of the implant with the superior and inferior vertebrae to determine if it is acceptable to ensure proper fusion and minimize subsidence.

### RELATED APPLICATION

The present application is a division of U.S. patent application Ser. No. 18/031,307 filed Apr. 11, 2023, which claims priority from PCT Application Serial Number PCT/US2022/049515, filed Nov. 10, 2022, which claims priority from U.S. Provisional Patent Application 63/280,246 filed Nov. 17, 2021, all of which are incorporated herein by reference in their entirety and made a part of this application.

### FIELD OF THE INVENTION

This invention relates to a method and apparatus for selecting a spinal orthopedic implant. More particularly, this invention relates to the use of Hounsfield Units from a patient specific CT scan to select and place an optimum spinal orthopedic implant with a surface area correlated to localized bone density measurements.

### BACKGROUND OF THE INVENTION

In a patient experiencing back problems associated with spinal vertebrae C1 through S1, surgical implantation of an intervertebral body fusion cage may be required to replace diseased or damaged vertebral discs. Typically, such interbody fusion cages use an allograft or autograft bone within the implant to fuse the bone of the vertebrae above the cage with the bone of the vertebrae below the cage. As used herein, “cage” and “implant” are synonymous. One such implant is typically used per intervertebral body space, but on occasion more than one may be needed within the same space. It also may be necessary to replace more than one diseased or damaged vertebral disc.

Following spinal fusion surgery, a decrease in the vertical height of the vertebral body space between the two adjacent vertebrae may occur prior to complete fusion of the bone of the superior vertebrae with the bone of the inferior vertebrae. This is known as subsidence. As a result, when a surgeon performs a fusion surgery, the surgeon attempts to restore the necessary vertical height using the selected intervertebral body cage. However, forces may prevent the complete height from being restored. When the allograft (consisting of cortical or cancellous bone tissue harvested from another human donor) or autograft (cortical or cancellous bone tissue harvested from the patient being treated) bone is used in the disc space to facilitate the bone fusion, a compressive force is placed on the bone graft due either to gravity or to the use of a fixation cage and supplemental fixation such as posterior pedicle screws to compress the two adjacent vertebrae against the bone graft. Human bones remodel using compressive force, a concept known as Wolff's law. Therefore, most surgeons want the bone graft to have a slight load on it following completion of the surgery, recognizing that this loading can reduce the effective vertical height of the operational level between the two adjacent vertebrae. Further, subsidence of the intervertebral body cage itself into the cortical bone at the interface of the cage and the two vertebrae reduces the effective height of the vertebral body space as the integrity of the bone at the contact point of the cage with the two adjacent vertebrae gives way to the hardness of material properties of the cage. Thus, surgeons accept that some settling occurs due to the subsidence but that loss in height can be compensated by the rest of the vertebrae anatomy as the patient heals. Unfortunately, too much subsidence or reduction of height can lead to non-fusion of the bone graft with one or both adjacent vertebrae, fracture of the cage, or even additional deterioration or disease of adjacent levels of vertebral body.

Several prior art systems have attempted to control subsidence through the material properties in the cage or implant. Early versions of interbody cages were made of carbon fiber, and then titanium, and also PEEK (polyetheretherketone). PEEK allowed manufacturers to attempt to match the modulus of elasticity of the bone graft. The thinking was that more of the compression would be taken by the bone graft and not shielded by the cage. Subsidence, in that case, would be between the bone graft and the endplates of each vertebra. The endplate of a vertebra is the transition region where the vertebrae and the disc interface. A vertebral endplate is commonly comprised of two layers: (1) a cartilaginous layer (also called cartilaginous endplate that fuses with the natural disc; and (2) a thin layer of cortical bone (also called the endplate) that attaches to the vertebral bone. Beneath the endplate and throughout the inner volume of the vertebra is cancellous bone, which is generally softer and arrayed in a randomized trabecular pattern. The surface area of the bone graft within the cage against the endplate is generally larger than the contact surface of the cage against the endplate. As such, an effort was made to make the contact area of the bone graft window within the cage as large as possible to maximize the amount of bone graft contacting the endplate of the vertebrae to absorb the vertical load.

With the advent of manufacturing technologies using titanium alloy, designs were then made to manipulate the density of the cage by varying the effective porosity to achieve the same effect. Nexxt Spine (Noblesville, Indiana) released the Matrixx family of cages in 2017, with a fully porous structure to provide a modulus of elasticity engineered to be compatible to PEEK devices.

More recently, U.S. Pat. No. 10,779,954, teaches the use of a dual energy x-ray absorptiometry (DEXA) scan to select a preferred spinal implant. A DEXA scan is a means of measuring bone density by directing two x-ray beams with different energy levels at the target bone of a patient's diseased or injured site. When the soft-tissue absorption is subtracted out, the bone mineral density (BMD) can be determined for each beam from the absorption of the beam by the bone. Using only the DEXA number (BMD) for the target site, U.S. Pat. No. 10,779,954 teaches the surgeon to select one of three implants provided in a kit. The problem with this technology is that it only uses DEXA values to select an implant and ignores the importance of adequate surface area between the contact surface of the implant or cage with the endplate of the vertebrae to maximize the likelihood of a proper fusion.

Thus, there is a need in the industry for a method to select a preferred implant or cage that considers the density of the endplates of the target bone and the adequacy of the contacting surface area between the cage and the endplate of the target bone to (1) minimize subsidence and (2) maximize the likelihood of an acceptable fusion of the cage, bone graft and endplates of contacting vertebrae.

### SUMMARY OF THE INVENTION

The apparatus of the present invention is a spinal implant for insertion between the endplates of adjacent vertebrae. It would normally replace a herniated or damaged disc. The implant comprises a circumscribing wall that defines an interior hollow portion. The wall comprises a superior surface defining a superior opening and an inferior surface defining an inferior opening. The implant further comprises a first arching portion which extends inwardly from the wall and upwardly towards the superior surface resulting in a decreased size of the superior opening. In addition, the implant may include a second arching portion which extends inwardly from the wall and downwardly towards the inferior surface decreasing the size of the inferior opening. The amount of arching inwardly by the first and second arching portions thereby defines the superior and inferior surface areas contacting the endplates.

In the method of the present invention a spinal fusion implant is selected for insertion between the endplates of adjacent vertebrae. A radio density scan (e.g., computed tomography (CT)) of the endplate of the vertebrae adjacent the herniated disc to be replaced is obtained. Using the radio density scan, an image of the contact surface of the selected implant is placed on an image of the endplate of the radiodensity scan. A Hounsfield Unit score is then determined for the contact surface of the image of the endplate using the radio density scan. A Hounsfield Unit is well known in the art and is a quantitative measurement of radiodensity. It may be referred to hereafter occasionally as “HU.” The Hounsfield Unit score is then compared with the corresponding area from which the Hounsfield Unit was obtained to generate a Hounsfield Parameter (“HP”) value. If an acceptable HP is achieved, confirmation is then made that the superior and inferior openings and surface areas of the implant are adequate to permit fusion of the adjacent vertebrae bone with the bone graft placed inside the implant with minimal subsidence.

One object of the present invention is to provide an implant having a sufficient contact surface with the endplate of the contact vertebrae, and a method for the selection of same, to minimize interbody subsidence and maintain adequate vertical height.

Another object of the present invention is to provide an implant having sufficiently large superior and inferior openings to permit fusion of the contact endplates of the vertebrae with the bone graft, and a method for the selection of same.

Thus, the present invention satisfies a complex tradeoff that surgeons have tried to address in the past: provide an anatomically conforming implant suitable for the patient that generates enough surface area to minimize subsidence yet still provides enough open space at the superior and inferior openings within the implant for the endplates to fuse with the bone graft placed within the interior volume of the implant, thereby maximizing the chance for proper fusion. Such a result is achieved through a preoperative plan that includes a routine radio density scan capable of measuring Hounsfield Unit scores.

Other and further objects, features, and advantages of the present invention will be apparent from the following description of the present invention, given for the purpose of disclosure, and taken in conjunction with the accompanying drawings. It is to be understood that the following detailed description and the accompanying drawings are not to be taken in a limiting sense.

### BRIEF DESCRIPTION OF THE DRAWINGS

FIGS. 1A, 1B, and 1C are perspective views of three implants of the present invention.

FIGS. 2A, 2B, and 2C are top plan views of the three implants of the present invention shown in corresponding FIGS. 1A, 1B, and 1C, respectively.

FIGS. 3A, 3B, and 3C are cross-sectional views of the three implants shown in FIGS. 1A, 1B, and 1C taken along lines 3A, 3B, 3C in FIGS. 2A, 2B, and 2C.

FIG. 4A illustrates a diseased or herniated disc between two vertebrae.

FIG. 4B illustrates one implant of the present invention surgically inserted between two vertebrae.

FIG. 4C illustrates two implants of the present invention within a single interbody spacing between adjacent vertebrae.

FIG. 5 is a CT radiodensity scan of the endplate of the target vertebrae of the patient.

FIG. 6 is a schematic of four boundary regions tested in five human cadaver bones.

FIG. 7 is a schematic of the mechanical indenture testing device.

FIG. 8 is a schematic of five indenter test sites at the four boundary regions in FIG. 6.

FIG. 9 is a graph of the format for the results of testing of the present invention.

FIGS. 10A-10D are graphs of test results of the four boundary regions for the L2 lumbar vertebra level.

FIGS. 11A-11D are graphs of test results of the four boundary regions for the L3 lumbar vertebra level.

FIGS. 12A-12D are graphs of test results of the four boundary regions for the L4 vertebra level.

FIGS. 13A-13D are graphs of test results of the four boundary regions for the L5 vertebra level.

FIG. 14 is a summary bar graph of test results for the parameter “Span” for each of the four boundary regions.

FIGS. 15A and 15B are representative images of the calculation of the Hounsfield Parameter for the inner boundary region of interest based on the Hounsfield Unit score and area from a CT scan.

FIGS. 16A and 16B are representative images of the calculation of the Hounsfield Parameter for the middle boundary region of interest based on the Hounsfield Unit score and area from a CT scan.

FIGS. 17A and 17B are representative images of the calculation of the Hounsfield Parameter for the outer boundary region of interest based on the Hounsfield Unit score and area from a CT scan.

FIGS. 18A and 18B are representative image of the calculation of the Hounsfield Parameter for the periphery boundary region of interest based on the Hounsfield Unit score and area from a CT scan.

FIG. 19A is a mapping of the Hounsfield Parameter value results for all regions of interest and indenter test sites on a CT radiodensity scan of the endplate of the target vertebrae from cadaveric testing in the present invention.

FIG. 19B is a modified mapping of the Hounsfield Parameter value results for all regions of interest and indenter test sites on a CT radiodensity scan of the endplate of the target vertebra of a patient using the present invention.

FIG. 20 is a CT radiodensity scan of the endplate of the target vertebrae of the patient overlayed with the mapping of the patient specific Hounsfield Parameter values from FIG. 19B and overlayed with a representative image of a small anterior implant.

FIG. 21 is a CT radiodensity scan of the endplate of the target vertebrae of the patient overlayed with the mapping of the patient specific Hounsfield Parameter values from FIG. 19B and overlayed with a representative image of the same size small anterior implant from FIG. 20.

FIG. 22 is a CT radiodensity scan of the endplate of the target vertebrae of the patient overlayed with the mapping of the patient specific Hounsfield Parameter values from FIG. 19B and overlayed with a representative image a large anterior implant.

FIG. 23 is a CT radiodensity scan of the endplate of the target vertebrae of the patient overlayed with the mapping of the patient specific Hounsfield Parameter values from FIG. 19B and overlayed with a representative image of a lateral implant.

### DETAILED DESCRIPTION OF THE PREFERRED EMBODIMENTS

The present invention now will be described more fully hereinafter with reference to the accompanying drawings, in which at least some preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

Characteristics and advantages of the present disclosure and additional features and benefits will be readily apparent to those skilled in the art upon consideration of the following detailed description of exemplary embodiments of the present disclosure and referring to the accompanying figures. It should be understood that the description herein and appended drawings, being of example embodiments, are not intended to limit the claims of this patent application or any patent or patent application claiming priority hereto. On the contrary, the intention is to cover all modifications, equivalents and alternatives falling within the spirit and scope of this disclosure or any appended claims. Many changes may be made to the particular embodiments and details disclosed herein without departing from such spirit and scope.

In showing and describing preferred embodiments in the appended figures, common or similar elements are referenced with like or identical reference numerals or are apparent from the figures and/or the description herein. The figures are not necessarily to scale and certain features and certain views of the figures may be shown exaggerated in scale or in schematic in the interest of clarity and conciseness.

As used herein and throughout various portions (and headings) of this patent application, the terms “invention”, “present invention” and variations thereof are not intended to mean every possible embodiment encompassed by this disclosure or any particular claim(s). Thus, the subject matter of each such reference should not be considered as necessary for, or part of, every embodiment hereof or of any particular claim(s) merely because of such reference. The terms “coupled”, “connected”, “engaged”, “attached”, and the like, and variations thereof, as used herein and in the appended claims are intended to mean either an indirect or direct connection or engagement. Thus, if a first device couples to a second device, that connection may be through a direct connection, or through an indirect connection via other devices and connections.

Certain terms are used herein and in the appended claims to refer to particular components. As one skilled in the art will appreciate, different persons may refer to a component by different names. The use of a particular or known term of art as the name of a component herein is not intended to limit that component to only the known or defined meaning of such term (e.g. bar, member, connector, rod, cover, panel, bolt, screw, and pin). Further, this document does not intend to distinguish between components that differ in name but not function. Also, the terms “including”, “comprising”, and “having” are used herein and in the appended claims in an open-ended fashion, and thus should be interpreted to mean “including, but not limited to” Further, reference herein and in the appended claims to components and aspects in a singular tense does not necessarily limit the present disclosure or appended claims to only one such component or aspect, but should be interpreted generally to mean one or more, as may be suitable and desirable in each particular instance.

As used herein, the terms “elongated” and variations thereof mean having an average length that is greater than its average width. As used herein, the terms “substantially”, “generally” and variations thereof means and includes (i) completely, or 100%, of the referenced parameter, variable or value, and (ii) a range of values less than 100% based upon the typical, normal or expected degree of variation or error for the referenced parameter, variable or value in the context of the particular embodiment or use thereof, such as, for example, 90-100%, 95-100% or 98-100%.

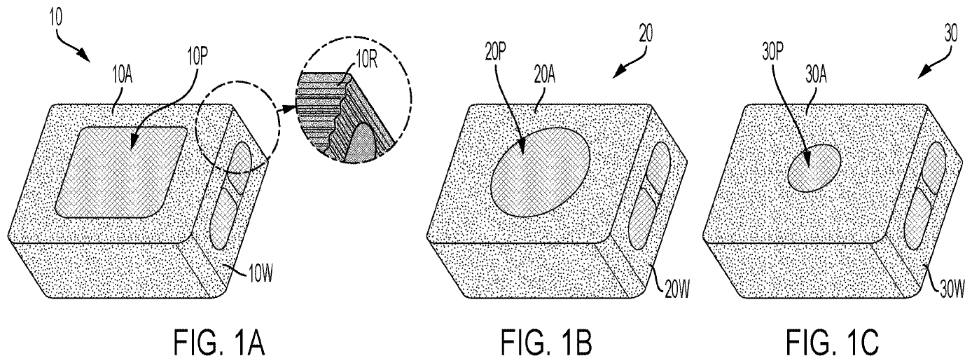

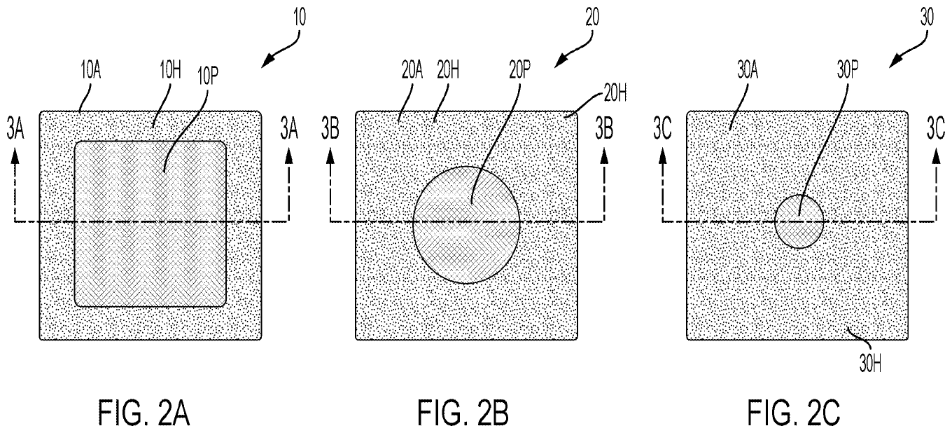

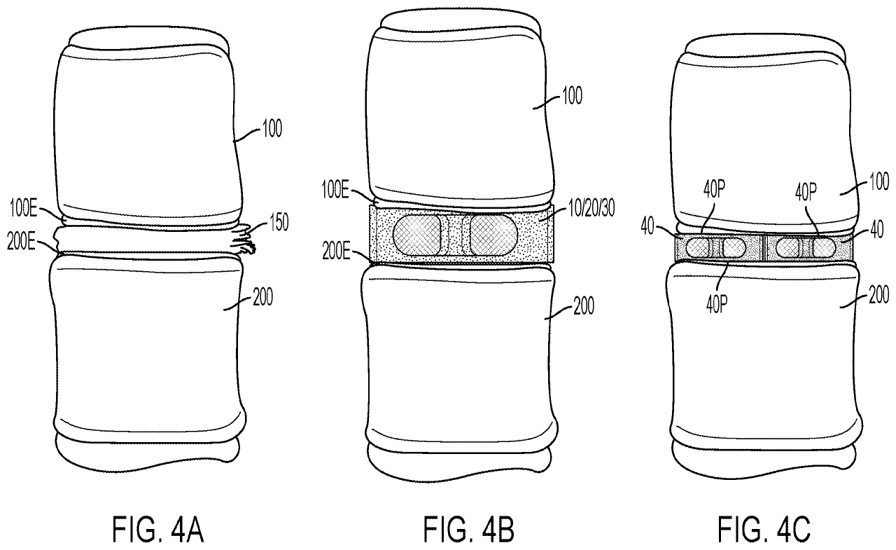

Referring now to FIGS. 1-4, three intervertebral implants or cages Oct. 20, 1930 are shown for insertion between two vertebrae 100/200, replacing a diseased or herniated disc 150 (FIG. 4A). The various implants are configured in various widths, depths, heights and lordotic angle to accommodate a significant sector of the patient populations; however, such dimensions are not significant for purposes of this disclosure.

Referring still to FIGS. 1-4, each implant includes a circumscribing wall 10W/20W/30W defining a contact surface area 10A, 20A and 30A of each implant. Contact surfaces 10A/20A/30A engage endplate 100E of the superior contacting vertebrae 100. Similarly, the opposite sides of each implant's circumscribing wall Oct. 20, 1930 includes a similar contact surface 10B/20B/30B to engage the endplate 200E of the inferior contacting vertebrae 200. As a result, each implant Oct. 20, 1930 includes an opening 10P/20P/30P defined within the contact surfaces 10A/10B/20A/20B/30A/30B of the implants.

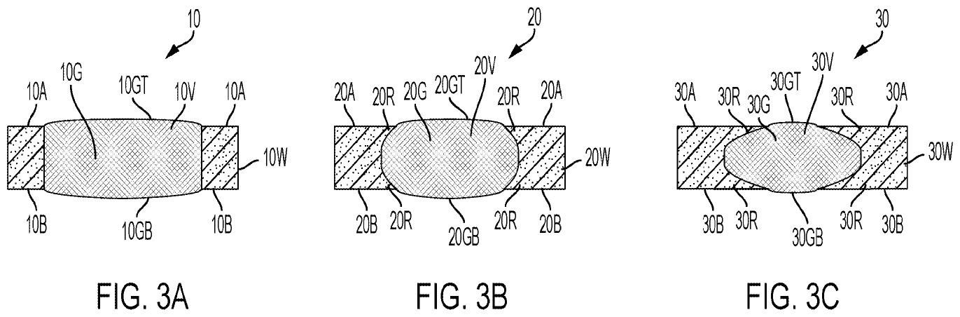

Referring now to FIGS. 3B and 3C, walls 20W and 30W include progressively arching portions 20R/30R which span inwardly within the interior 20V/30V reducing the corresponding openings 20P and 30P. During surgery the surgeon places allograft or autograft bone 10G/20G/30G within the interior volume 10V/20V/30V of implants Oct. 20, 1930 selected. The surgeon compacts bone graft 10G/20G/30G within the interior 10V/20V/30V, preferably leaving the superior and inferior surfaces 10GT/20GT/30GT and 10GB/20GB/30GB of the bone graft 10G/20G/30G slightly elevated so that endplates 100E/200E contact the bone graft before contacting surfaces 10A/10B/20A/20B/30A/30B of the implants.

If the selected implant has too small a bone graft window or opening 30P, for example, to permit proper fusion to occur, the surgeon may elect to place more than one implant 40, as shown in FIG. 4C. Such would provide two openings 40P (or more depending on the number of implants 40 used) and more porous holes through which fusion may occur. To confirm that adequate support is provided to minimize subsidence two templates would be placed on the CT radiodensity scan and the CT scan would generate a HU range for two templates. With such templates providing adequate support to minimize subsidence the surgeon is then satisfied with multiple openings 40P for fusion integrity.

In a patient with normal bone, implant 10 is selected preferrable having a thinner wall 10W with a contact surface 10A defined by the thickness of wall 10W, thereby defining the bone graft window through opening 10P that may contact the endplate 100E/200E of vertebrae 100/200. Thus, the bone graft window and opening 10P are the same, as this is the window of bone graft that will contact endplates 100E/200E. As noted above, when the surgeon is preparing bone graft 10G he will compress the bone graft 10G within interior volume 10V but leaves a slight elevation of bone graft 10G for extending beyond openings 10P so that the implant 10 can be compressed between vertebrae 100/200 with minimal subsidence as the fusion healing occurs among endplates 100E, bone graft 10G and endplate 200E. Once fused, this bone graft forms an integral column of bone extending from within endplate 100E, through the fused bone graft 10G and into endplate 200E.

In less dense bone types with lower bone quality and integrity, the contact surface area of the cage or implant should increase so that the implant takes more of the load during fusion than would typically pass to the bone graft in a healthier patient using implant 10, thereby providing the opportunity for proper fusion and to minimize subsidence as well. To achieve this, reference is now made to implants 20 and 30 in FIGS. 1-3 as described above. To increase the contact surface area 20A/30A of implants 20 and 30 and redirect more load into the implant during fusion at least, implants 20/30 include arching portions 20R/30R which extend inwardly into the interior volume 20V/30V in the manner shown in FIGS. 3B and 3C. Such necessarily reduces the size of the bone graft window or openings 20P/30P and increases the size of contact surface areas 20A/30A of the implant. By increasing the size of implant contact surfaces 20A/30A, more compressive load is redirected to the implant due to the harder material. In other words, the contact surface areas 20A/30A of implants 20/30 that interact with the endplates 100E/200E can effectively spread the same compressive forces over more surface area. Thus, less subsidence will occur in this patient with less dense bone than would have occurred if an implant 10 had been used. Similarly, since more load is being taken by the implant, at least initially while the patient is healing, fusing among endplates 100E/200E and bone graft 20G/30G is permitted to occur under less harsh or stressful conditions, which is beneficial.

Implants Oct. 20, 1930 still include pores or openings 10H/20H/30H throughout the contact surfaces areas 10A/10B/20A/20B/30A/30B to allow adequate interaction of the vertebral body endplates 100E/200E with bone graft 10G/20G/30G, particularly the additional surface areas 20A/20B/30A/30B resulting from the use of the arching portions 20R/30R. In addition, implants Oct. 20, 1930 may include ridges 10R for additional bone retention. (See FIG. 1A detail).

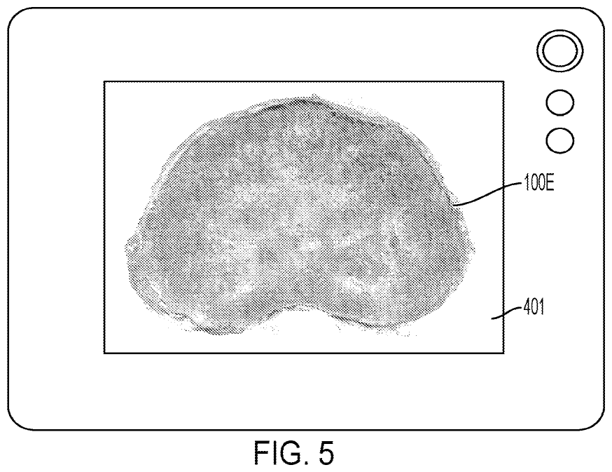

In practicing the present invention during the pre-operative phase, an image 401 of a CT radiodensity scan of the endplate 100E of the target vertebrae as shown in FIG. 5 is obtained. The CT radiodensity scan used is capable of generating HU. Such a CT radiodensity scan is well known in the art. A HU is a quantitative measurement of radiodensity of a defined area. It is used by radiologists in the interpretation of CT images. The absorption/attenuation coefficient of radiation within a tissue is used during CT reconstruction to produce a grayscale image as shown in FIG. 5. HU, also referred to as the CT unit, is determined with the CT scan based on a linear transformation of the baseline linear attenuation coefficient of the X-ray beam, where distilled water (at standard temperature and pressure) is arbitrarily defined to be zero HU and air defined as −1000 HU. The upper limits can reach up to 1000 HU for bones, 2000 HU for dense bones, and more than 3000 for metals like steel or silver. The linear transformation produces a Hounsfield scale that displays as gray tones. More dense tissue, with greater X-ray beam absorption, has positive values and appears bright; less dense tissue, with less X-ray beam absorption, has negative values and appears dark. See, Hounsfield Unit, NCBI Bookshelf, www.ncbi.nlm.nih.gov>books>NBK547721.

The image in FIG. 5 shows a typical cross section image of a vertebral body demonstrating the thin cortical bone layer at the periphery of the image and cancellous bone throughout the interior volume of the vertebra. The image 401 is generated from a CT radiodensity scan (as discussed above generating Hounsfield Units) of an image approximately halfway between the endplates of the target vertebrae, but an optimal image will generally be at least 10 mm within the endplates. The cortical layer will register HU values closer to 1000, while the interior volume of the vertebra consists of bone and air due to the porosity of the cancellous bone.

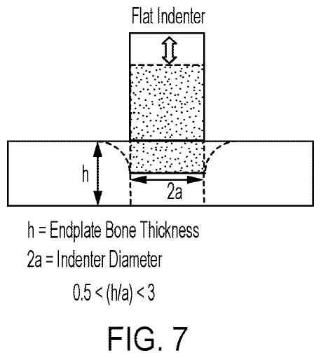

To confirm the accuracy of using HU as an indicator the following study was performed to attempt to correlate HU to certain mechanical properties of human cadaver vertebrae. Testing was conducted under dynamic conditions to establish a vertebral endplate map with resulting mechanical data correlated to HU. Cyclic indention testing was the primary test. The purpose was to establish a dynamic mechanical response to localized cyclic loading and to correlate the resulting mechanical parameters to the localized values of HU. A schematic of the indenture apparatus used is shown in FIG. 7. This apparatus permitted the orientation of the vertebral test location to be placed in a near perpendicular alignment with the loading axis (parallel with the test face of the apparatus).

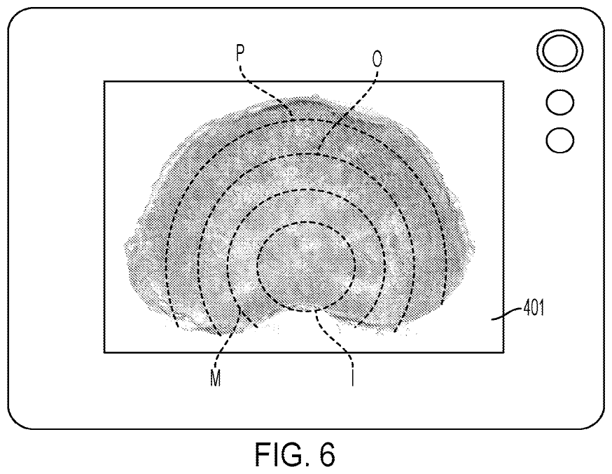

Five human cadaver lumbar segments from L2 to L5 between the ages of 40 and 80 were used. The vertebrae of each segment was devoid of soft tissue. Each vertebrate was subjected to a CT radiodensity scan with the resulting HU values from the four regions of interest (ROI) identified in FIG. 6 as inner (I), middle (M), outer (O), and periphery (P) and measured at the 50% level of the vertebral height as measured from the superior endplate, as well as the area (A) encompassed by the region measured in voxels. The term voxel is well known in the art and represents a value on a regular grid in 3D computer mapping, as a pixel does in 2D bitmapping.

The HU data was based upon calibration to a value of −1000 for an air environment. The regional HU values were adjusted by adding this baseline value to the reported HU values. This was performed for five human spines at lumbar levels L2, L3, L4 and L5. Finally, to account for the total area the resulting regional HU value per unit area was subjected to squaring. The resulting parameter encompassing both the HU and the area was termed the Hounsfield Parameter (HP). HP was used to establish correlations between HU and mechanical evaluation within the regions of interest according to the following equation:

$$HP = {[ ( \frac{{ROI}_{n + 1} + 1000}{{Area}_{n + 1}} ) - ( \frac{{ROI}_{n} + 1000}{{Area}_{n}} ) ]}^{2} \qquad Equation 1$$

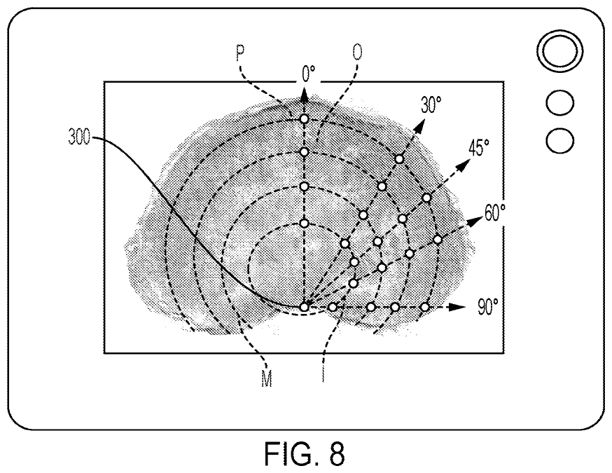

Referring to FIG. 8, the mechanical evaluation of the ROI is depicted. The location of each indenture test site is shown at the intersection of each boundary (inner, middle, outer, and periphery) and radial coordinates along 0°, 30°, 45°, 60°, and 90° vectors. Therefore, a total of 20 results test sites were located on each vertebra L2 through L5.

For each vertebra tested, the 20 test sites were subjected to cyclic fatigue loading. Referring still to FIG. 8, the posterior-central location 300 was used to normalize the resulting mechanical parameter data across vertebral bodies. This site has been identified with increased and uniform mechanical properties as it is adjacent to the spinal canal and thus, more protective in nature.

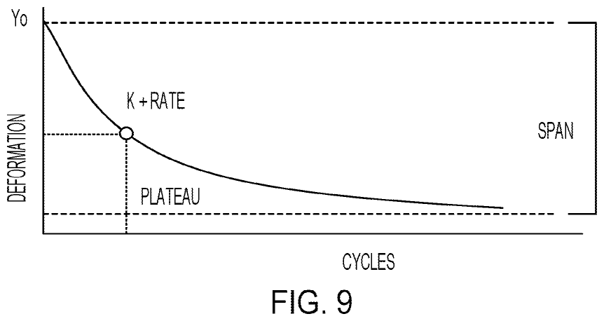

A cadaver vertebral body sample was prepared for each of the 20 test locations for each vertebra. For the indenture test each site was subjected to 250 cycles of compressive load from −2.5 N (Newtons) to −25 N at a rate of 1 Hz. Deformation changes over the applied load cycles were calculated for each cycle interval at each of the indentation sites for each vertebra. Normalization of the deformation data was performed as a percentage of the deformation seen at the reference point 300 for each vertebra. The deformation data for each test point was be plotted versus cycle number and subjected to non-linear regression. For each test site, a non-linear exponential regression was performed that provided clinically relevant parameters of Yo (Initial Deformation), Plateau (Asymptotic Deformation Limit), Span (Total Subsidence), Half Life (Number of cycles to achieve a 50% subsidence from Yo) and K (the deformation per unit cycle). The visual representation of the mathematical response is seen in FIG. 9.

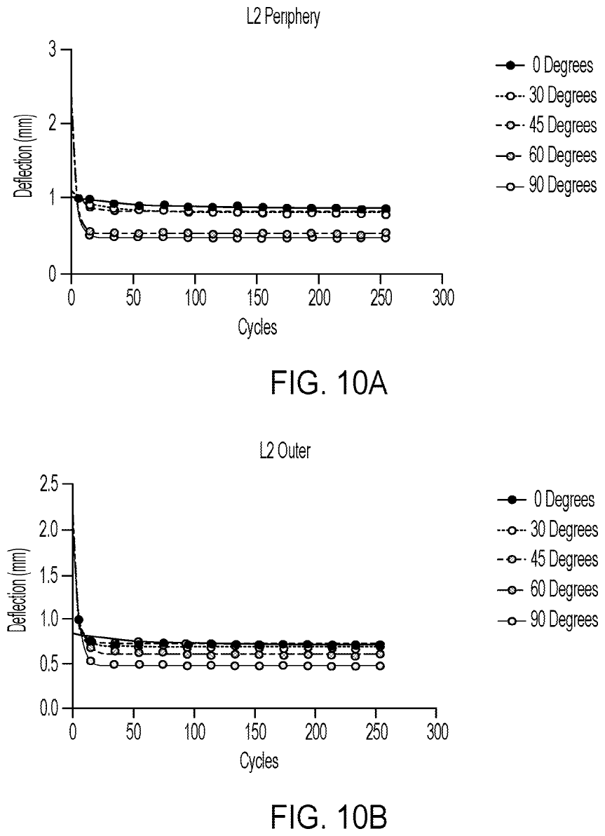

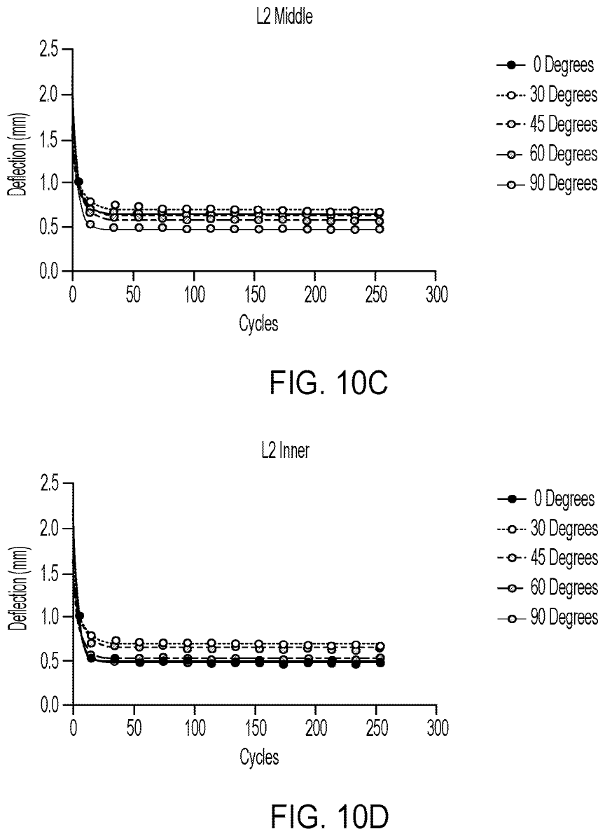

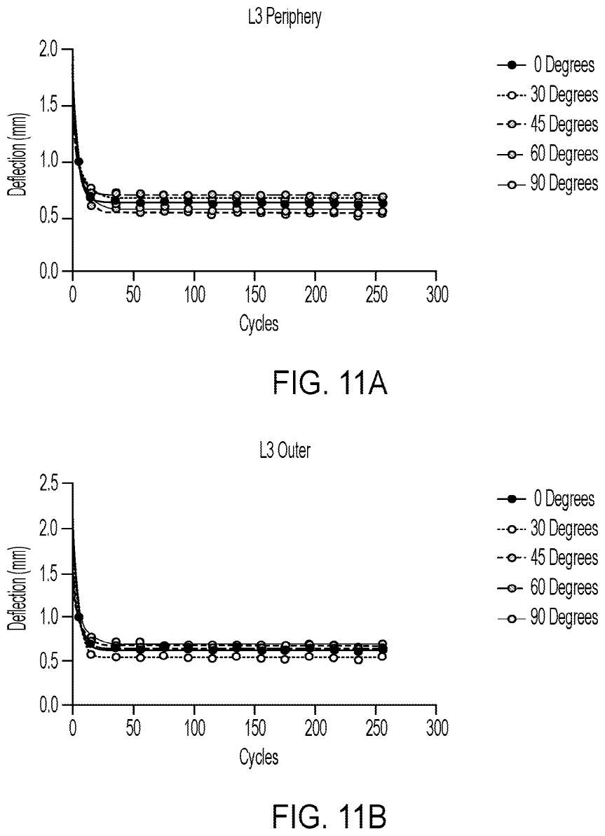

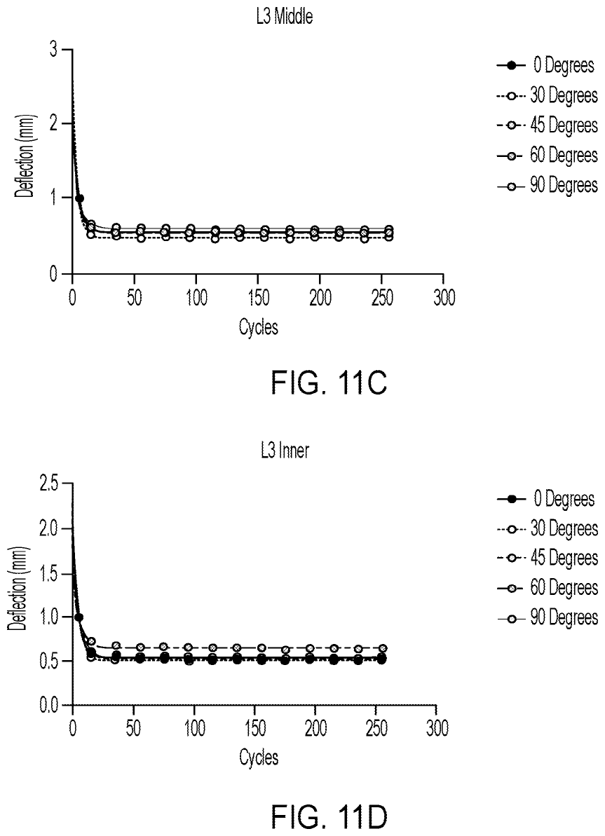

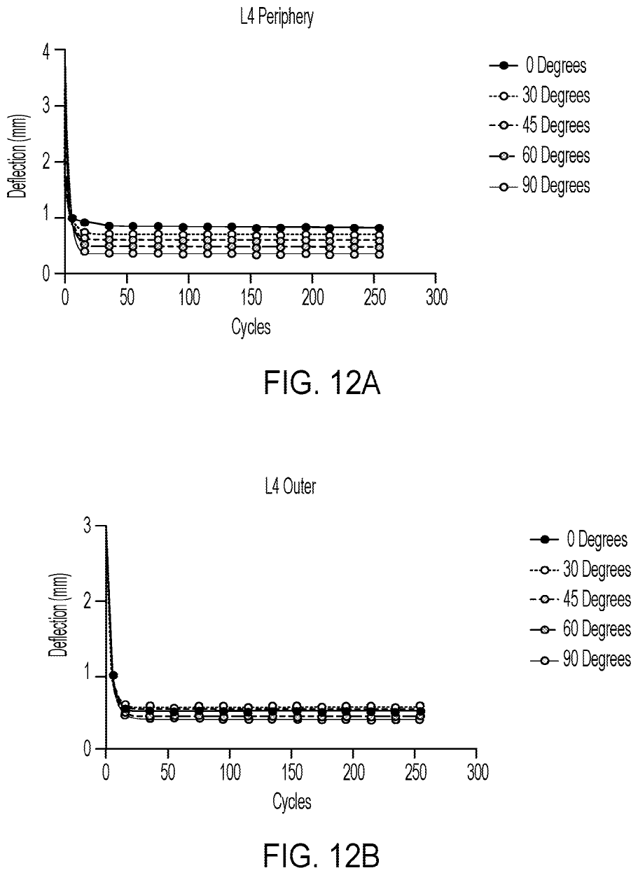

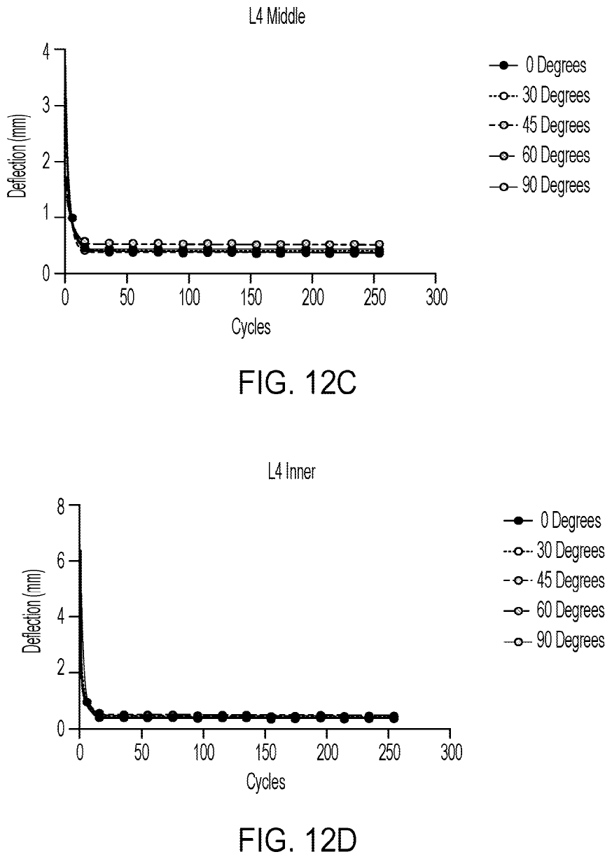

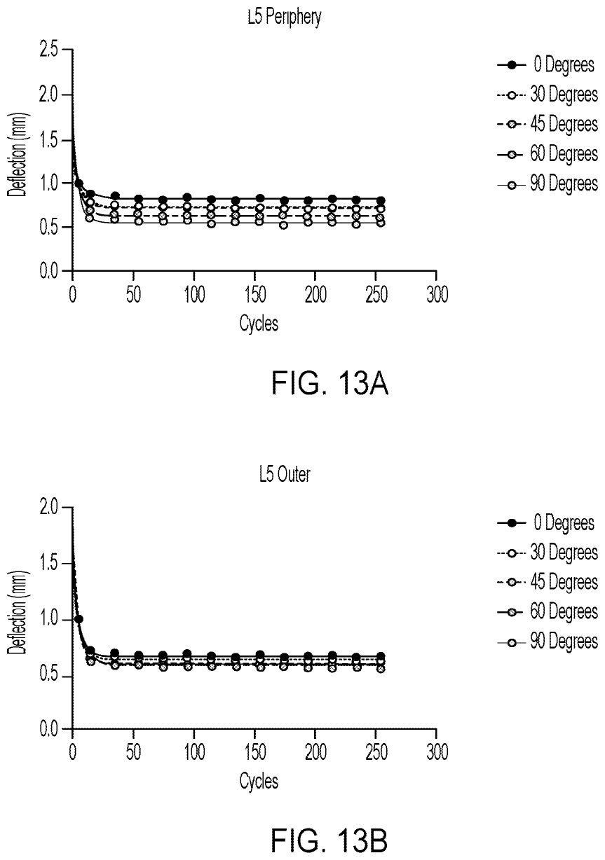

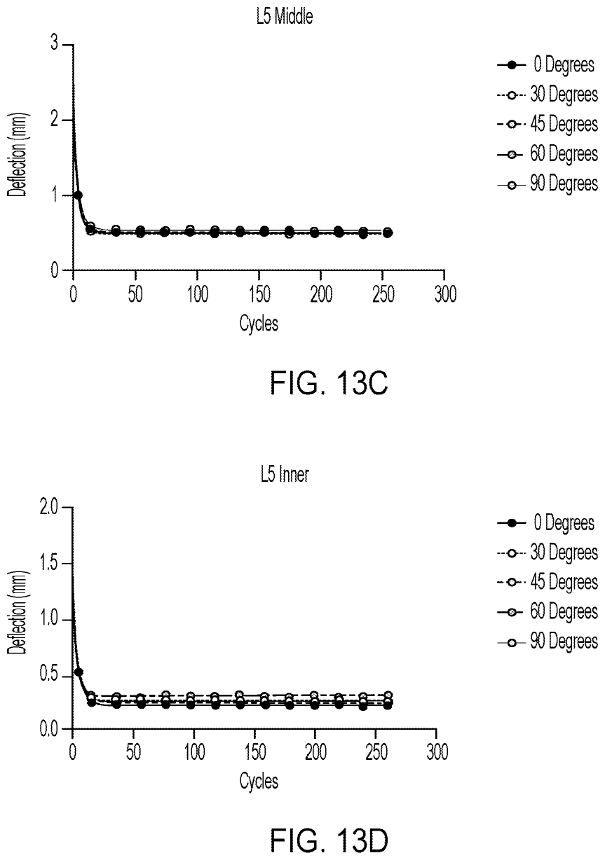

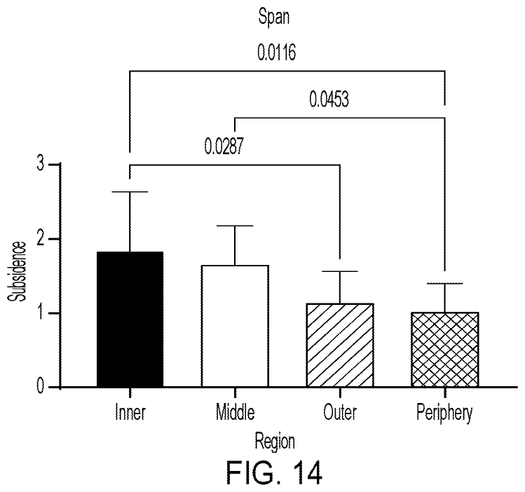

FIGS. 10A-10D illustrate the typical response curves for ROI sites for the four boundary regions (periphery, outer, middle, inner) for the L2 lumbar vertebra level. FIGS. 11A-11D illustrate the typical response curves for ROI sites for the four boundary regions (periphery, outer, middle, inner) for the L3 lumbar vertebra level. FIGS. 12A-12D illustrate the typical response curves for ROI sites for four the boundary regions (periphery, outer, middle, inner) for the L4 vertebra level. FIGS. 13A-13D illustrate the typical response curves for ROI sites for four boundary regions (periphery, outer, middle, inner) for the L5 vertebra level.

Referring to FIG. 14, a summary bar graph of the test results for the parameter “Span” (as defined in FIG. 9) is shown for each of the four boundary regions which indicates a correlation between subsidence and the measured span variable.

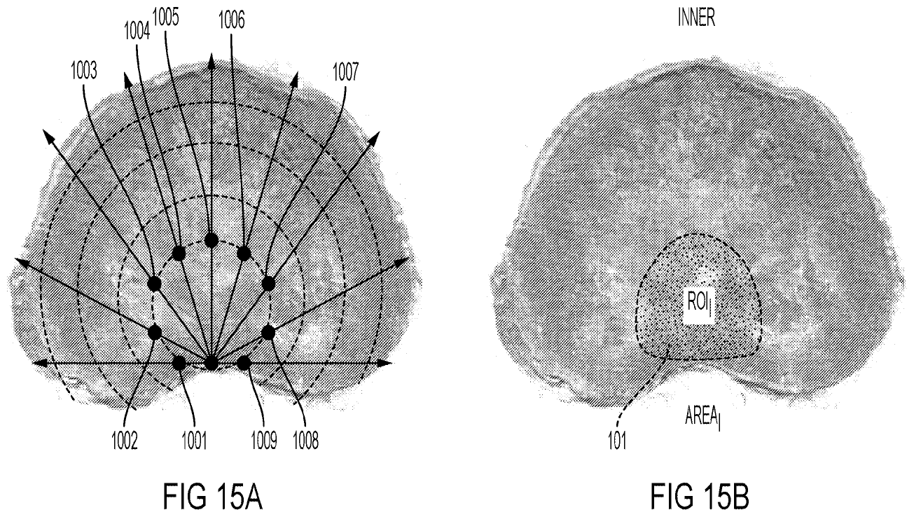

Next, HP is calculated using equation 1 above. One method of determining HP from cadaveric testing results is referenced by FIGS. 15-18. Referring to FIGS. 15A and B, the representative image for the calculation of HP for the inner boundary is based on the HU score of the inner ROI boundary encompassing points 1001 through 1009 over area 101, which is the area of the ROI from the CT scan that corresponds to the HU score given. HP was calculated using equation 1 above based on the mapping of the results of the CT scan.

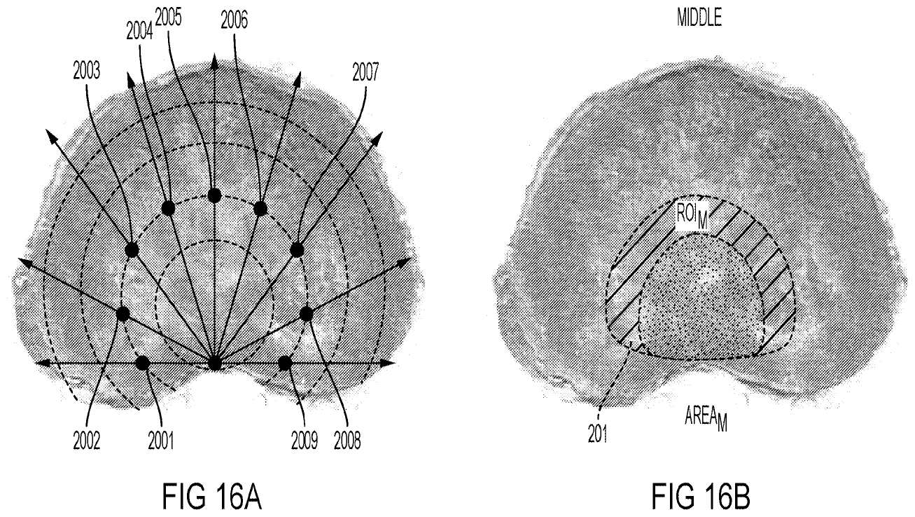

Referring to FIGS. 16A and B, the representative image for the calculation of HP for the middle boundary is based on the HU score of the middle ROI boundary encompassing points 2001 through 2009 area 201, which is the area of the ROI from the CT scan that corresponds to the HU score given. HP was calculated using equation 1 above based on the mapping of the results of the CT scan for HU and area 201 but subtracting the results for HU and area 101 from the inner region.

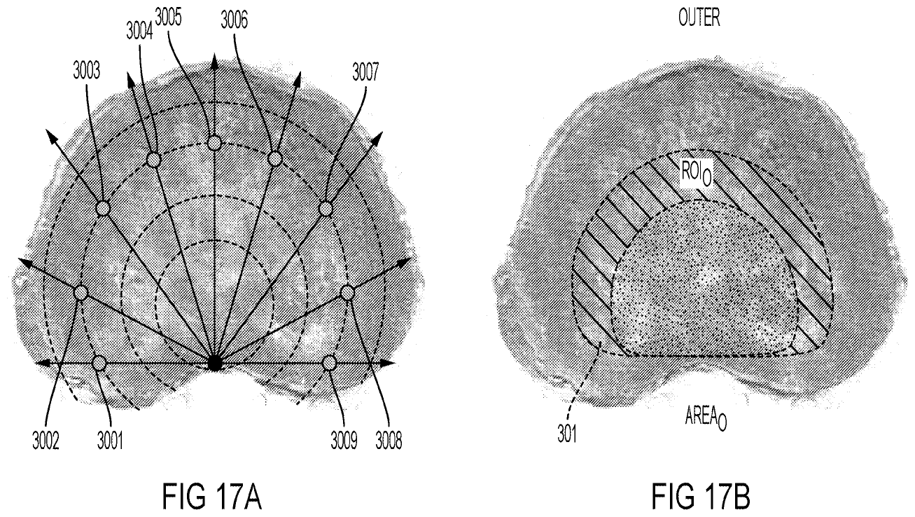

Referring to FIGS. 17A and B, the representative image for the calculation of HP for the outer boundary is based on the HU score of the outer ROI boundary encompassing points 3001 through 3009 over area 301, which is the area of the ROI from the CT scan that corresponds to the HU score given. HP was calculated using equation 1 above based on the mapping of the results of the CT scan for HU and area 301 but subtracting the results for HU and area 201 from the middle region.

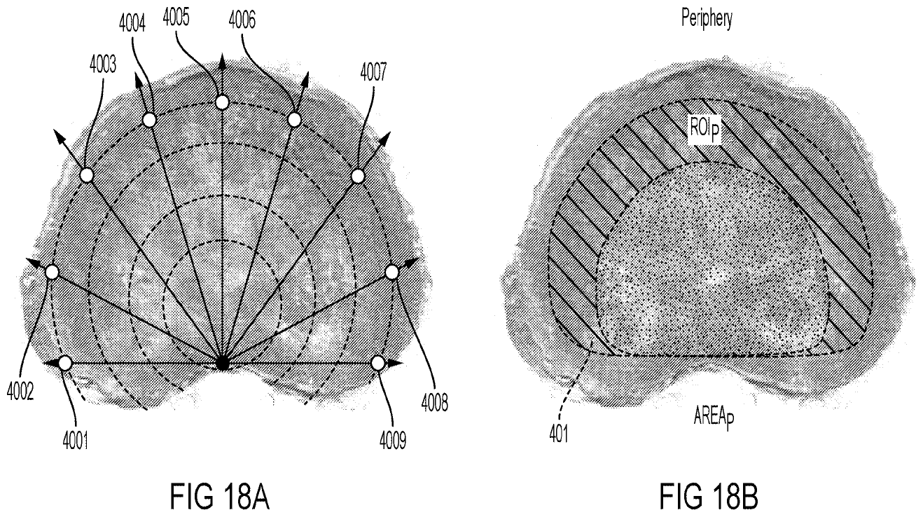

Referring to FIGS. 18A and B, the representative image for the calculation of HP for the periphery boundary is based on the HU score of the periphery ROI boundary encompassing points 4001 through 4009 over area 401, which is the area of the ROI from the CT scan that corresponds to the HU score given. HP was calculated using equation 1 above based on the mapping of the results of the CT scan for HU and area 401 but subtracting the results for HU and area 301 from the outer region.

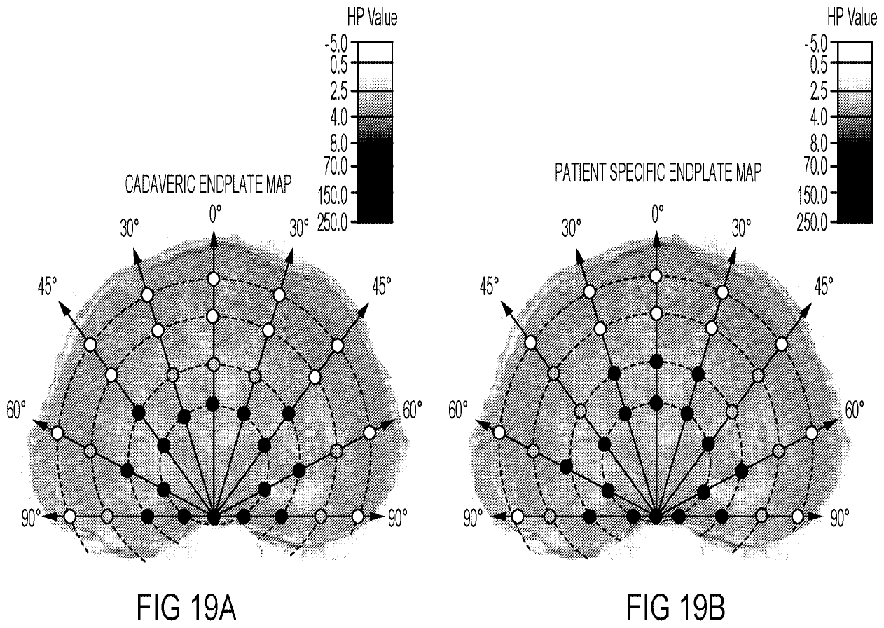

Referring to FIG. 19A, the results of the correlation analysis indicate that a significant association between the HU parameter, as computed to isolate an ROI, can be associated with the mechanical response within the ROI based on several parameters extracted under dynamic loading. Referring still to FIG. 19A, the final results from the cadaveric testing inform a gradation shading scale corresponding to increasing risk of subsidence for a given boundary. HP values are shown from a low-risk region of −5.0 through 0.5, a medium-risk region of 0.5 through 8.0 and a high-risk region from 8.0 to 250. The points within each boundary are then plotted on a cadaver endplate map at the 90°, 60°, 45°, 30°, and 0° vector locations mirrored about the midline, on the assumption that left and right sides have the same properties. Then, referring to FIG. 19B, an actual pre-operative CT scan for a specific patient is shown with the gradation shading scale along the vector radial lines modified based again on the measured HP as described above. For each ROI and vertebral level, changes in the HP values for the specific patient compared to HP values from the testing modify the gradation of each region to have higher or lower risk of subsidence. Modifications to the gradation may also change individual points within a region to different risk level.

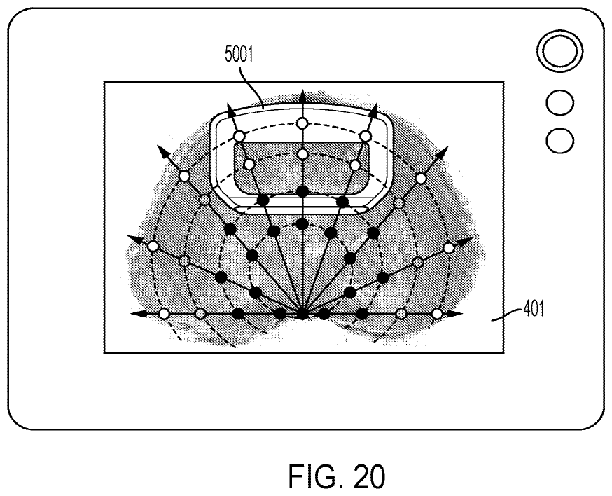

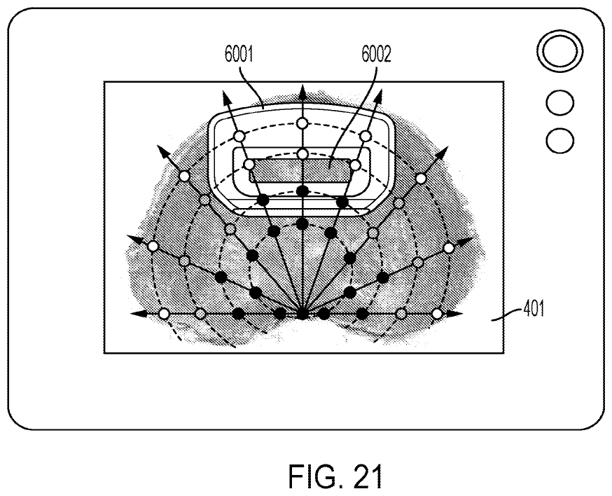

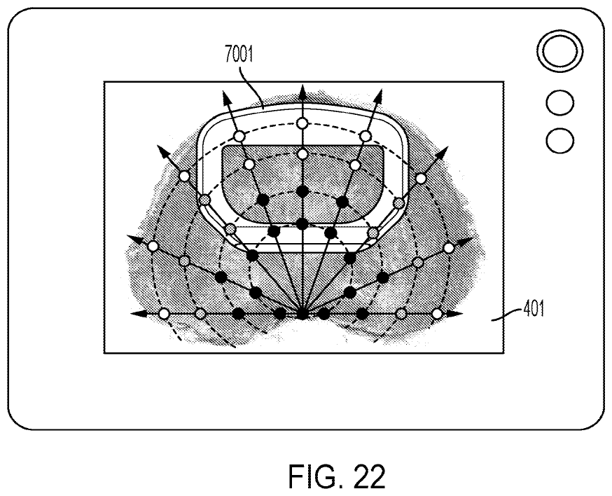

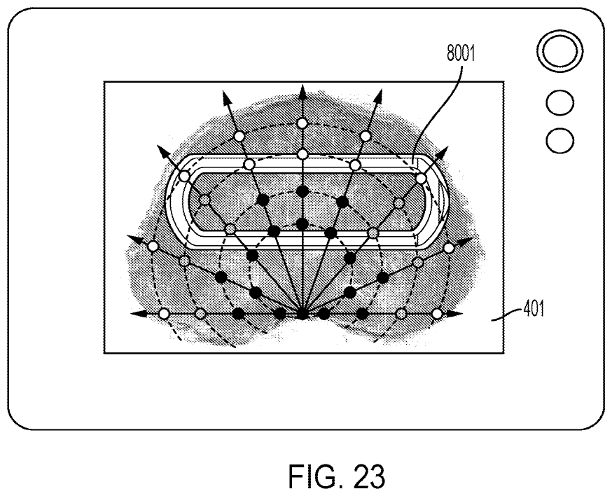

Applying the patient specific endplate map of the CT scan as shown in FIG. 19B, the surgeon is provided several templates over the mapped interactive CT endplate for this patient as shown in FIGS. 20-23. For example, FIG. 20 illustrates a default smaller anterior implant 5001 with a nominal surface area. FIG. 21 illustrates the same smaller anterior implant 6001 of FIG. 20 but with a larger surface area by reducing the size of the interior graft window opening 6002 to cover more of the low-risk subsidence points. FIG. 22 illustrates a scaled up anterior implant 7001 with the same surface area as the implant in FIG. 20 but covering different low-risk subsidence points. And finally FIG. 23 illustrates a lateral implant 8001 with the same surface area as FIG. 20 but also covering different low-risk subsidence points. Using these selections, the surgeon would select the optimum implant taking into account the HP readings for the patient and the area of the interior opening to provide for the best opportunity for proper fusion.

It should be recognized that an implant with larger outer dimensions and a large opening 10P may have the same surface contact area as a smaller implant with smaller outer dimensions and a smaller opening. Thus, in patients with inadequate bone density, the surgeon may wish to place various templates on the CT scan with various outer dimensions and opening sizes but similar surface contact surfaces. This is shown by comparing various templates as shown in FIGS. 20-23. These results indicate a definite relationship between bone strength, as assessed by HU values and calculated HP as set forth herein.

What is claimed is:

1. A spinal implant for insertion between the endplates of adjacent vertebrae comprising:
a circumscribing wall member defining an interior hollow portion;
said wall member comprising:
a superior surface defining a superior opening,
an inferior surface defining an inferior opening,
a first arching portion extending inwardly from the wall member and upwardly towards the superior surface decreasing the size of the superior opening; and
a second arching portion extending inwardly from the wall member and downwardly towards the inferior surface decreasing the size of the inferior opening,

2. The spinal implant of claim 1 wherein the amount of arching inwardly by said first and second arching portions defining the superior and inferior openings being determined by an acceptable Hounsfield Parameter value from a radiodensity scan of the endplates of the vertebrae contacting the superior and inferior surfaces of between about −5.0 and 8.0.

3. The spinal implant of claim 2 wherein the Hounsfield Parameter value is between about −5.0 and 0.5.

4. The spinal implant of claim 1 wherein said wall member includes a porous structure.

5. A spinal implant for insertion between the endplates of adjacent vertebrae comprising:
a circumscribing wall member defining an interior hollow portion;
said wall member comprising:
a superior surface defining a superior opening,
an inferior surface defining an inferior opening, and
a first arching portion extending inwardly from the wall member and upwardly towards the superior surface decreasing the size of the superior opening,
wherein the amount of arching inwardly by the first arching portion defining the superior opening being determined by an acceptable Hounsfield Parameter value determined from a radiodensity scan of the endplates of the vertebrae contacting the superior surface.

6. The spinal implant of claim 5 further comprising a second arching portion extending inwardly from the wall member and downwardly towards the inferior surface decreasing the size of the inferior opening wherein the amount of arching inwardly by the second arching portion defining the inferior opening being determined by an acceptable Hounsfield Parameter value determined from a radiodensity scan of the endplates of the vertebrae contacting the inferior surface of between about −5.0 and 8.0.

7. The spinal implant of claim 6 wherein the Hounsfield Parameter value is between about −5.0 and 0.5.

8. The spinal implant of claim 5 wherein said wall member includes a porous structure.

9. A spinal implant for insertion between the endplates of adjacent vertebrae comprising:
a circumscribing wall member defining an interior hollow portion;
said wall member includes porous structure and further comprises:
a superior surface defining a superior opening, and
an inferior surface defining an inferior opening;
a first arching portion extending inwardly from the wall member and upwardly towards the superior surface decreasing the size of the superior opening wherein the amount of arching inwardly by the first arching portion defining the superior opening being determined by an acceptable Hounsfield Parameter value determined from a radiodensity scan of the endplates of the vertebrae contacting the superior surface; and
a second arching portion extending inwardly from the wall member and downwardly towards the inferior surface decreasing the size of the inferior opening wherein the amount of arching inwardly by the second arching portion defining the inferior opening being determined by an acceptable Hounsfield Parameter value determined from a radiodensity scan of the endplates of the vertebrae contacting the inferior surface.

10. The spinal implant of claim 9 wherein the Hounsfield Parameter value is between about −5.0 and 8.0.

11. The spinal implant of claim 10 wherein the Hounsfield Parameter value is between about −5.0 and 0.5.
